(19) United States
(12) Patent Application Publication
(10) Pub. No.: US 2023/0193184 A1
(43) Pub. Date: Jun. 22, 2023

(54) CELL CULTURE APPARATUS

(71) Applicant: TOYODA GOSEI CO., LTD., Kiyosu-shi (JP)

(72) Inventors: Seitaro TAKI, Kiyosu-shi (JP); Sayaka HINO, Nagoya-shi (JP)

(21) Appl. No.: 18/060,170

(22) Filed: Nov. 30, 2022

(30) Foreign Application Priority Data
Dec. 21, 2021 (JP) 2021-206725

(51) Int. Cl. C12M 1/42; C12M 3/00; C12M 1/12

(52) U.S. Cl. CPC C12M 35/08; C12M 23/44; C12M 25/02; C12M 23/48

(57) ABSTRACT

A cell culture apparatus includes: a cylindrical member that includes a body cylindrical portion and a culture membrane arranged at an opening end portion of the body cylindrical portion; and a cylindrical covering member that is fitted to the body cylindrical portion and includes (i) a first cylindrical portion which covers an outer circumferential surface of the body cylindrical portion when fitted to the cylindrical member and (ii) a second cylindrical portion which is adjacent to the first cylindrical portion in an axial direction of the covering member and forms a container with the culture membrane serving as a bottom surface when fitted to the cylindrical member, and the first cylindrical portion includes a first part in which a length in the axial direction is a first length and a second part in which the length is a second length shorter than the first length.

### CROSS REFERENCE TO RELATED APPLICATIONS

This application claims priority based on Japanese Patent Application No. 2021-206725 filed on Dec. 21, 2021, the entire disclosure of which is hereby incorporated by reference.

### BACKGROUND

### Field

The present disclosure relates to cell culture apparatuses.

### Related Art

Recently, cell culture has been performed on both surfaces of a cell culture membrane. Hence, cell culture devices for performing culture on both surfaces are proposed (see, for example, International Publication No. WO 2012/045368 and “Kanto Chemical Co., Inc./Product Inforation/ad-MED Vitrigel (registered trademark) 2”, “online” “searched on Nov. 9, 2021”, the Internet<URL:https://products.kanto.co.jp/web/index.cgi?c=t_product_table&pk=55>). Since in the cell culture device of ad-MED Vitrigel (registered trademark) 2, cell culture is performed on a surface directed to the outside of an insert in both surfaces of a cell culture membrane, a cylindrical option ring is fitted to the outside of the insert. The option ring is attached and detached with tweezers.

However, in the option ring of ad-MED Vitrigel (registered trademark) 2, it may be difficult to attach and detach the option ring to the insert.

### SUMMARY

The present disclosure is able to be realized as an aspect below.

According to one aspect of the present disclosure, a cell culture apparatus is provided. The cell culture apparatus includes: a cylindrical member that includes a body cylindrical portion and a culture membrane arranged at an opening end portion of the body cylindrical portion; and a cylindrical covering member that is fitted to the body cylindrical portion and includes (i) a first cylindrical portion which covers an outer circumferential surface of the body cylindrical portion when fitted to the cylindrical member and (ii) a second cylindrical portion which is adjacent to the first cylindrical portion in an axial direction of the covering member and forms a container with the culture membrane serving as a bottom surface when fitted to the cylindrical member, and the first cylindrical portion includes a first part in which a length in the axial direction is a first length and a second part in which the length is a second length shorter than the first length. In the aspect described above, the first cylindrical portion includes the first part and the second part the lengths of which are different from each other. Hence, the covering member is moved with a part near the second part serving as a fulcrum from a posture where the center axis of the covering member is inclined with respect to the center axis of the body cylindrical portion to bring the second part into contact with the cylindrical member before the first part such that the center axis of the covering member is parallel to the center axis of the cylindrical member. In this way, it is possible to fit the covering member to the cylindrical member.

### BRIEF DESCRIPTION OF THE DRAWINGS

FIG. 1 is a perspective view of a cell culture apparatus;

FIG. 2 is a cross-sectional view of the cell culture apparatus;

FIG. 3 is a cross-sectional view of the cell culture apparatus in a fitted state;

FIG. 4 is a perspective view of a grasping device;

FIG. 5 is a diagram illustrating a method of fitting a covering member to a cylindrical member;

FIG. 6 is a flowchart showing a cell culture step;

FIG. 7 is a schematic view illustrating the cell culture step; and

FIG. 8 is a perspective view of a cell culture apparatus according to a second embodiment.

### DETAILED DESCRIPTION

### A. First Embodiment

### A1. Configuration of Cell Culture Apparatus

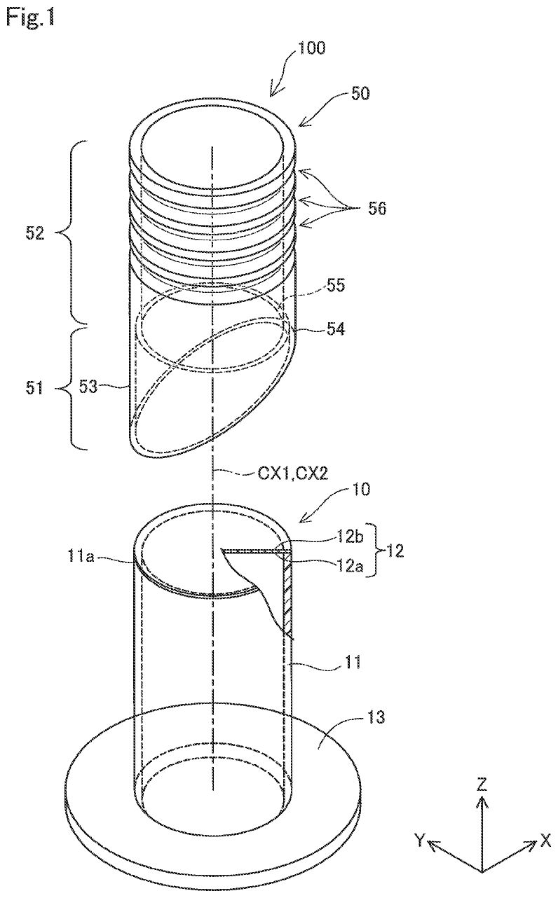

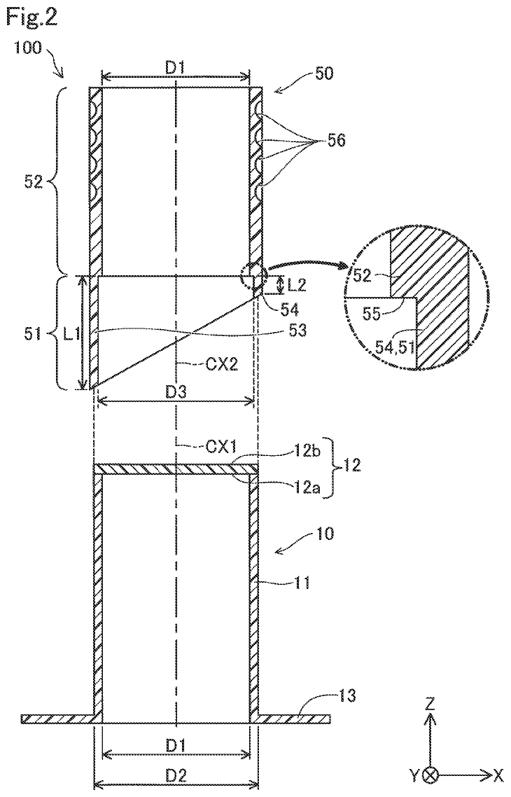

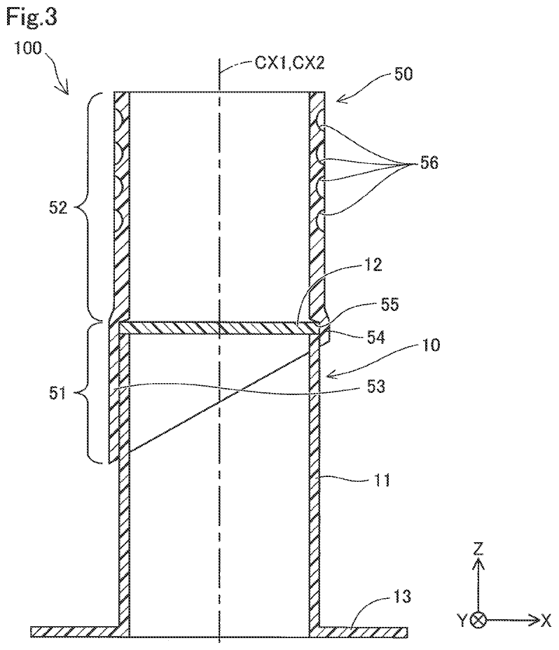

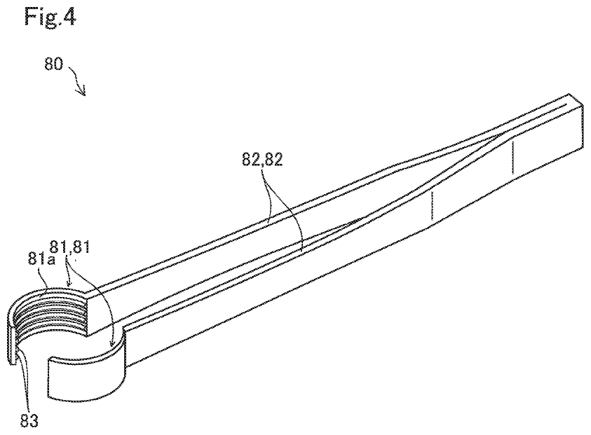

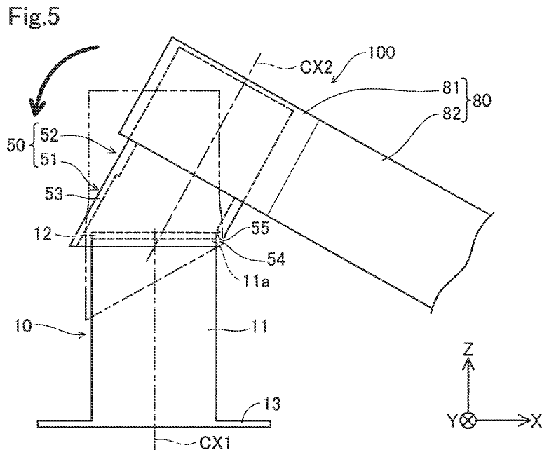

FIG. 1 is a perspective view of a cell culture apparatus 100. FIG. 2 is a cross-sectional view of the cell culture apparatus 100. FIG. 3 is a cross-sectional view of the cell culture apparatus 100 in a fitted state where a covering member 50 is fitted to a cylindrical member 10. FIG. 4 is a perspective view of a grasping device 80. FIG. 5 is a diagram illustrating a method of fitting the covering member 50 to the cylindrical member 10. in FIGS. 1 to 3 and 5, an X-axis, a Y-axis and a Z-axis are shown which are orthogonal to each other. The cell culture apparatus 100 is typically arranged such that a Z-direction is along a vertical direction and is used. Directions in which the arrows of the X-axis, the Y-axis and the Z-axis are directed respectively indicate positive directions along the X-axis, the Y-axis and the Z-axis. The positive directions along the X-axis, the Y-axis and the Z-axis are assumed to be +X-direction, +Y-direction and +Z-direction, respectively. Directions opposite to the directions in which the arrows of the X-axis, the Y-axis and the Z-axis are directed respectively indicate negative directions along the X-axis, the Y-axis and the Z-axis. The negative directions along the X-axis, the Y-axis and the Z-axis are assumed to be −X-direction, +Y-direction and −Z-direction, respectively. When whether the direction is positive or negative is not defined, directions along the X-axis, the Y-axis and the Z-axis are referred to as the X-direction, the Y-direction and the Z-direction, respectively. The same is true for drawings and description to be given below. The +Z-direction may be referred to as an upward direction, and the −Z-direction may be referred to as a downward direction.

The cell culture apparatus 100 includes the cylindrical member 10, the covering member 50 and the grasping device 80 FIG. 4). As shown in FIG. 1, the cylindrical member 10 includes a body cylindrical portion 11, a culture membrane 12 and a flange 13. For example, the body cylindrical portion 11 and the flange 13 are integrally molded of a resin material. As the resin material, high-density polyethylene, polypropylene, an ABS resin, an acrylic resin, polycarbonate, polystyrene, polyester or the like may be used. The flange 13 is in a circular shape, and extends in a radial direction from one end in an axial direction which is a direction along the center axis CX1 of the cylindrical member 10. The culture membrane 12 is arranged at an opening end portion 11a of the body cylindrical portion 11. Specifically, the culture membrane 12 is arranged at the opening end portion 11a of two end portions of the cylindrical member 10 in the axial direction on a side opposite to the end at which the flange 13 is formed. The circular culture membrane 12 is joined to the opening end portion 11a of the body cylindrical portion 11 so as to cover the opening end portion 11a of the body cylindrical portion 11. The culture membrane 12 includes a first surface 12a which is directed inward of the body cylindrical portion 11 and a second surface 12b which is a surface on a side opposite to the first surface 12a. In a cell culture step, cells are cultured on each of the first surface 12a and the second surface 12b.

The culture membrane 12 is a membrane which serves as a footing for cell culture when the cell culture is performed. As the culture membrane 12, a porous membrane which includes a large number of pores penetrating in the direction of thickness of the membrane and is formed of synthetic polymer or a porous membrane which is formed of natural polymer may be used. Specific examples of the porous membrane include a track etching membrane, a nanofiber membrane, a collagen vitrigel membrane, a honeycomb membrane, a porous polyurethane membrane and the like. Examples of the porous polyurethane membrane include a porous polyurethane membrane which is manufactured by a manufacturing method including a first step and a second step to be subsequently described and the like. The first step is a step of forming, on a substrate, a layer of a polyurethane raw material which is uncured. The second step is a step of supplying water vapor to an exposed surface moved away from the substrate in the layer of the polyurethane raw material formed on the substrate to facilitate the curing of the polyurethane raw material in the layer of the polyurethane raw material and to form a porous shape with a plurality of recesses and projections in the exposed surface.

The covering member 50 is cylindrical. In the cell culture step, the covering member 50 is used while fitted to the cylindrical member 10. The covering member 50 is flexible and formed of a material having a low coefficient of dynamic friction with respect to the cylindrical member 10. Specifically, as the material having a low coefficient of dynamic friction, low density polyethylene (LDPE), polypropylene, tetrafluoroethylene-perfluoroether copolymer (PFA) or the like may be used. The covering member 50 may be formed by performing, on the surface of a flexible material, surface treatment for decreasing the coefficient of dynamic friction. Specifically, the covering member 50 may be formed by coating the surface of the flexible material with diamond-like carbon (DLC), 2-methacryloyloxyethylphosphorylcholine (MPC) polymer or the like. In this way, as will be described in detail later, in the process of attaching and detaching the covering member 50 to and from the cylindrical member 10, workability is able to be enhanced.

The covering member 50 includes a first cylindrical portion 51 and a second cylindrical portion 52. As shown in FIG. 3, the first cylindrical portion 51 is a part of the body of the covering member 50 which covers the outer circumferential surface of the body cylindrical portion 11 when the covering member 50 is fitted to the cylindrical member 10. The second cylindrical portion 52 is a part of the body of the covering member 50 which does not cover the outer circumferential surface of the body cylindrical portion 11 when the covering member 50 is fitted to the cylindrical member 10. The second cylindrical portion 52 is adjacent to the first cylindrical portion 51 in an axial direction along the center axis CX2 of the covering member 50. When the second cylindrical portion 52 is fitted to the cylindrical member 10, the second cylindrical portion 52 forms a container in which the second cylindrical portion 52 is a side surface and the culture membrane 12 is a bottom surface. As shown in FIG. 2, the first cylindrical portion 51 includes a first part 53 in which a length in the axial direction is a first length L1 and a second part 54 in which a length in the axial direction is a second length L2. The second length L2 is shorter than the first length L1. In this way, as will be described in detail later, in the process of attaching and detaching the covering member 50 to and from the cylindrical member 10, workability is able to be enhanced. The first part 53 and the second part 54 are opposite each other in the radial direction orthogonal to the axial direction. As the first cylindrical portion 51 extends, along a circumferential direction with the axial direction serving as a center, from the first part 53 toward the second part 54, the length in the axial direction is gradually decreased. In other words, in the present embodiment, when the end surface of the first cylindrical portion 51 of the covering member 50 is seen from the outside in the radial direction, the end surface is inclined with respect to the center axis CX2 whereas when the end surface is seen along the center axis CX2, the shape of the end surface is an ellipse.

The covering member 50 further includes a step portion 55 in an inner circumferential surface. The thickness of the first cylindrical portion 51 in the radial direction is less than the thickness of the second cylindrical portion 52 in the radial direction. The outside diameter of the first cylindrical portion 51 is equal to the outside diameter of the second cylindrical portion 52. The inside diameter D3 of the first cylindrical portion 51 is greater than the inside diameter D1 of the second cylindrical portion 52. Then, in a boundary between the inner circumferential surface of the first cylindrical portion 51 and the inner circumferential surface of the second cylindrical portion 52, the step portion 55 is formed by a difference between the inside diameter of the first cylindrical portion and the inside diameter of the second cylindrical portion 52. The step portion 55 is a surface which is formed in the circumferential direction in the boundary between the first cylindrical portion 51 and the second cylindrical portion 52. In the boundary, the step portion 55 protrudes inward in the radial direction from the inner circumferential surface of the first cylindrical portion 51. In this way, as will be described in detail later, in the process of fitting the covering member 50 to the cylindrical member 10, it is possible to easily position the covering member 50 with respect to the cylindrical member 10. The inside diameter D1 of the second cylindrical portion 52 is equal to the inside diameter D1 of the body cylindrical portion 11. In this way, in the cell culture step, an area over which cells are cultured on the first surface 12a and an area over which cells are cultured on the second surface 12b are able to be made equal to each other. The case where “the inside diameter D1 of the second cylindrical portion 52 is equal to the inside diameter D1 of the body cylindrical portion 11” is not limited to a case where the inside diameter of the second cylindrical portion 52 matches with the inside diameter of the body cylindrical portion 11 but may be a case where a difference between the inside diameter of the second cylindrical portion 52 and the inside diameter of the body cylindrical portion 11 is in a range of about plus or minus 10% of the inside diameter of the body cylindrical portion 11.

In the present embodiment, the inside diameter D1 of the body cylindrical portion 11 is about 6 mm, and the outside diameter D2 thereof is about 9 mm. The inside diameter D3 of the first cylindrical portion 51 is less than the outside diameter D2 of the body cylindrical portion 11 by about a length equal to or greater than 0.1 mm and equal to or less than 0.3 mm. Hence, the covering member 50 is extended to increase its diameter and is fitted to the cylindrical member 10. In this way, the covering member 50 is able to be fitted to the cylindrical member 10 so as to be in intimate contact therewith. The thickness of the first cylindrical portion 51 is about equal to or greater than 0.3 mm and equal to or less than 2 mm. In this way, it is possible to satisfactorily maintain the flexibility of the first cylindrical portion 51, and thus workability is able to be enhanced in the process of attaching and detaching the covering member 50 to and from the cylindrical member 10. When a flexible material is used, there is a tendency that as its thickness is increased, the flexible material is more unlikely to be deformed. Hence, the thickness of the first cylindrical portion 51 is in the range described above, and thus it is possible to satisfactorily maintain the flexibility.

The covering member 50 further includes, in the outer circumferential surface thereof, a plurality of groove portions 66 which extend in the circumferential direction. The plurality of groove portions 56 are formed by being recessed from the outer circumferential surface of the second cylindrical portion 52 toward the center axis CX2. In this way, as will be described in detail later, convex portions 83 included in the grasping device 80 are able to be fitted into the groove portions 56, and thus it is possible to stably grasp the cylindrical member 10 with the grasping device 80. Even when the grasping device 80 is not used, tips of a device such as tweezers are hooked on the groove portions 56, and thus it is possible to easily grasp the covering member 50 with the device.

FIG. 4 is a perspective view of the grasping device 80. The grasping device 80 is a device for grasping the second cylindrical portion 52 of the covering member 50. The grasping device 80 includes a pair of grasping portions 81, 81 and a pair of plate portions 82, 82. The pair of plate portions 82, 82 are plate-shaped, and in the direction of a long side, end portions on a side opposite to end portions at which the pair of grasping portions 81, 81 are arranged are joined to each other. In the pair of plate portions 82, 82, in a state where no external force is applied, as shown in FIG. 4, the pair of grasping portions 81, 81 are separated from each other. Then, when an external force is received, the pair of plate portions 82, 82 are reversibly deformed so as to approach each other. In this way, an operator holds the pair of plate portions 82, 82 in a state where the second cylindrical portion 52 is sandwiched between the pair of grasping portions 81, 81, and deforms the grasping device 80 to be able to grasp the covering member 50 with the grasping device 80.

When the second cylindrical portion 52 is grasped, the pair of grasping portions 81, 81 are opposite each other through the second cylindrical portion 52 in the radial direction of the second cylindrical portion 52. Each of the pair of grasping portions 81, 81 includes a grasp inner surface 81a which is a curved surface along the outer circumferential surface of the second cylindrical portion 52 (FIG. 1). In this way, it is possible to stably grasp the cylindrical member 10. In the grasp inner surface 81a, a plurality of convex portions 83 extending in the circumferential direction are formed in positions corresponding to the groove portions 56 included in the second cylindrical portion 52. Specifically, the convex portions 83 protrude outward from the grasp inner surface 81a along the radial direction. Intervals between the plurality of convex portions 83 are the same as intervals between the plurality of groove portions 56. In the present embodiment, the grasp inner surface 81a is covered with a sheet member, and the sheet member and the convex portion 83 are integrally molded. The sheet member and the convex portion 83 are formed of a material which has a high coefficient of static friction with respect to the covering member 50. The coefficient of static friction of the material of which the sheet member and the convex portion 83 are formed is preferably greater than 1. As the material having a high coefficient of static friction, isobutylene-isoprene rubber (IIR), natural rubber, ethylene-propylene rubber (EPDM), vinylidene fluoride rubber (FKM) or the like is able to be used. In this way, it is possible to stably grasp the covering member 50 with the grasping device 80.

As shown in FIG. 5, when the covering member 50 is fitted to the cylindrical member 10, the covering member 50 is first inclined with respect to the cylindrical member 10 installed such that the center axis CX2 of the covering member 50 is inclined with respect to the center axis CX1 of the cylindrical member 10. The covering member 50 is grasped by the grasping device 80. Then, before the first part 53, the inner circumferential surface of the second part 54 is brought into intimate contact with the outer circumferential surface of the cylindrical member 10. Here, since the covering member 50 includes the step portion 55, the opening end portion 11a of the body=cylindrical portion 11 is caused to abut on the step portion 55, and thus it is possible to easily position the covering member 50 with respect to the cylindrical member 10.

Then, the covering member 50 is moved in the direction of an arrow with a part near the second part 54 serving as a fulcrum such that the center axis CX2 of the covering member 50 is parallel to the center axis CX1 of the cylindrical member 10. As indicated by chain double-dashed lines, the covering member 50 is moved until the opening end portion 11a of the body cylindrical portion 11 abuts on the step portion 55. Likewise, since the covering member 50 includes the step portion 55, the opening end portion 11a of the body cylindrical portion 11 is able to be caused to abut on the step portion 55, and thus it is possible to easily position the covering member 50 with respect to the cylindrical member 10.

Since the first cylindrical portion 51 includes the second part 54 and the first part 53 the lengths of which are different from each other, by the procedure described above, it is possible to fit the covering member 50 to the cylindrical member 10. In the fitting method described above, an area of the inner circumferential surface of the first cylindrical portion 51 which slides against the outer circumferential surface of the cylindrical member 10 in the process of moving the covering member 50 is limited to a very small part of the inner circumferential surface in the vicinity of the first part 53. Hence, it is possible to reduce an area in which friction occurs between the inner circumferential surface of the covering member 50 and the outer circumferential surface of the cylindrical member 10 at the time of fitting. Therefore, it is possible to reduce a force necessary for fitting the covering member 50, and thus it is possible to easily fit the covering member 50, with the result that workability is able to be enhanced.

As shown in FIG. 3, the first cylindrical portion 51 of the covering member 50 is fitted to the cylindrical member 10 in a state where the first cylindrical portion 51 is deformed such that the outside and inside diameters thereof are increased. Here, the covering member 50 is flexible, and the thickness of the first cylindrical portion 51 is less than the thickness of the second cylindrical portion 52, with the result that the flexibility of the second cylindrical portion 52 is satisfactory. Hence, the covering member 50 is easily fitted, and in a fitted state, the covering member 50 is able to be in intimate contact with the first cylindrical portion 51.

When the covering member 50 is removed from the cylindrical member 10, the procedure described above is performed in reverse order. Specifically, the covering member 50 is first moved with the part near, the second part 54 serving as the fulcrum such that the center axis CX2 of the covering member 50 is inclined with respect to the center axis CX1 of the cylindrical member 10. After the first part 53 is moved away from the body cylindrical portion 11, the covering member 50 is moved such that the second part 54 is moved away from the body cylindrical portion 11. Even when the covering member 50 is removed from the cylindrical member 10, as in the case where the covering member 50 is fitted, it is possible to reduce the force necessary for fitting the covering member 50, and thus it is possible to easily remove the covering member 50, with the result that workability is able to be enhanced.

### A2. Cell Culture Method Using Cell Culture Apparatus

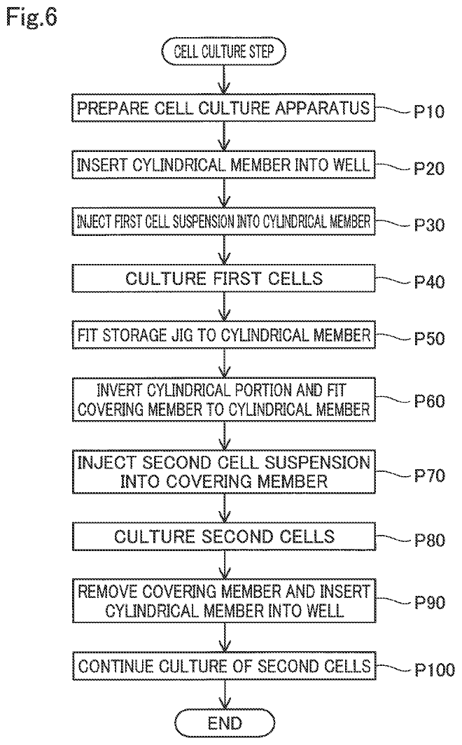

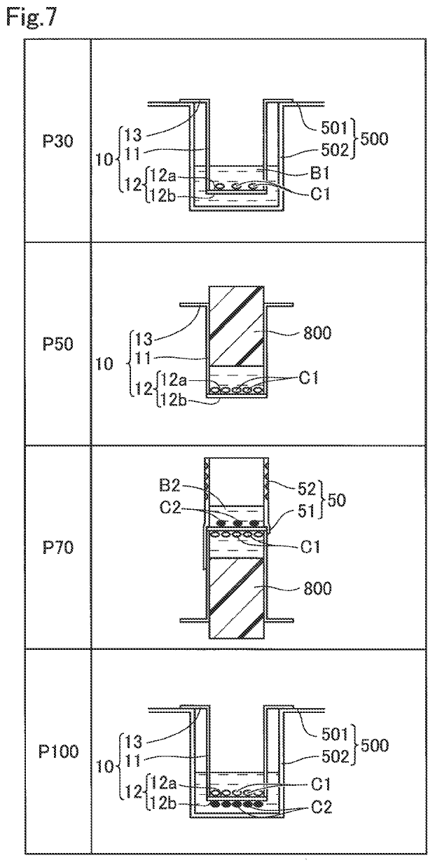

FIG. 6 is a flowchart showing the cell culture step for culturing cells on both surfaces of the culture membrane 12 using the cell culture apparatus 100. FIG. 7 is a schematic view which shows steps and states of the cell culture apparatus 100 such that the steps and the states correspond to each other and illustrates the cell culture step. In a step P10 shown in FIG. 6, the cell culture apparatus 100 is prepared. In a step P20, the cylindrical member 10 is inserted into the well 502 (FIG. 7) of a well plate 500 (FIG. 7). Specifically, as shown in the step P30 of FIG. 7, in a posture of the cylindrical member 10 taken by inverting the posture shown in FIG. 1, the flange 13 is locked on the upper surface 501 of the well plate 500 coupled to the well 502. In this way, the cylindrical member 10 is suspended within the well 502.

In the step P30 shown in FIG. 6, a first cell suspension B1 which includes first cells C1 and a culture solution for the first cells is injected into the cylindrical member 10. The first cell suspension B1 is injected to seed the first cells C1 on the first surface 12a of the culture membrane 12. In the present embodiment, the culture solution is also injected into the well 502. After the completion of this operation, the lid of the well plate 500 is closed. In a step P40, as necessary, the culture solution is appropriately replaced, and culture is performed until the first cells C1 are fixed on the first surface 12a of the culture membrane 12. The culture is performed within an incubator in which the temperature, the humidity and the CO₂ concentration are constant and which is a stable environment. In this way, the cells are able to be cultured in a state close to physiological conditions.

In a step P50, a storage jig 800 (FIG. 7) for storing the culture solution within the body cylindrical portion 11 is fitted to the cylindrical member 10. As shown in the step P50 of FIG. 7, the storage jig 800 is flexible and is formed into a cylindrical shape. The outside diameter of the storage jig 800 is large enough to be in intimate contact with the inner circumferential surface of the body cylindrical portion 11. The storage jig 800 is inserted into the body cylindrical portion 11 such that the center axis of the storage jig 800 is along the center axis CX1 of the cylindrical member 10. The tip of the storage jig 800 in a downward direction in which the storage jig 800 is inserted is inserted to such a position that the tip does not touch the culture membrane 12. In this way, even when in the subsequent step P60, the cylindrical member 10 is inverted, it is possible to store the culture solution within the body cylindrical portion 11. Hence, the culture of the first cells C1 is able to be continued.

In the step P60 shown in FIG. 6, the cylindrical member 10 is inverted, and the grasping device 80 is used to fit the covering member 50 to the outer circumferential surface of the cylindrical member 10. The covering member 50 is fitted, and thus a container is formed in which the culture membrane 12 is a bottom surface and the inner circumferential surface of the covering member 50 is a side surface and which stores the culture solution. Here, since the first cylindrical portion 51 is in intimate contact with the cylindrical member 10, it is possible to suppress the leakage of a second cell suspension B2 which is injected into the second cylindrica portion 52 in the subsequent step P70.

In the step P70, the second cell suspension B2 which includes second cells C2 and a culture solution for the second cells is injected into the covering member 50. As shown in the step P70 of FIG. 7, the second cell suspension B2 is injected to seed the second cells C2 on the second surface 12b of the culture membrane 12.

In a step P80 shown in FIG. 6, the culture solution is appropriately replaced as necessary and the second cells C2 are cultured until the second cells C2 included in the second cell suspension B2 are fixed on the second surface 12b of the culture membrane 12. The culture is performed within the incubator. When the culture is performed, for example, the culture is preferably performed in a wide-mouth container with a lid. In this way, it is possible to suppress the evaporation of the culture solution during the culture seeded into the covering member 50 and to suppress contamination during the culture. Here, since as described above, the inside diameter 1 of the second cylindrical portion 52 is equal to the inside diameter D1 of the body cylindrical portion 11, the area of the culture of the second cells C2 is able to be made equal to the area of the culture of the first cells C1.

In a step P90, the culture solution is extracted from the interior of the covering member 50, and the grasping device 80 is used to remove the covering member 50 from the cylindrical member 10. Thereafter, the cylindrical member 10 is inverted, and is inserted into the well 502 of the well plate 500 again. After the insertion of the entire cylindrical member 10, the lid of the well plate 500 is closed.

In a step P100, the culture of the second cells C2 is continued. The entire culture is performed within the incubator. Specifically, as shown in the step P100 of FIG. 7, the culture solution for the second cells C2 fixed on the second surface 12b is injected into the well 502, and the culture solution for the first cells C1 fixed on the first surface 12a is injected into the cylindrical member 10. The culture solution is replaced as necessary, and the culture is performed until the completion of a culture operation. When the culture of the second cells C2 is completed, the present cell culture step is completed. In the cell culture step, the cells are able to be cultured on each of the first surface 12a and the second surface 12b of the culture membrane 12.

In the first embodiment described above, the cell culture apparatus 100 including the cylindrical member 10 and the covering member 50 is provided. The covering member 50 includes; the first cylindrical portion 51 which covers the outer circumferential surface of the body cylindrical portion 11 when fitted to the body cylindrical portion 11; and the second cylindrical portion 52 which forms the container with the culture membrane 12 serving as the bottom surface. The first cylindrical portion 51 includes the first part 53 in which the length in the axial direction is the first length L1 and the second part 54 in which the length in the axial direction is the second length L2 shorter than the first length L1 Since the first cylindrical portion 51 includes the second part 54 and the first part 53 the lengths of which are different from each other, the covering member 50 is moved with the part near the second part 54 serving as the fulcrum from a state where the center axis CX2 of the covering member 50 is inclined with respect to the center axis CX1 of the cylindrical member 10 to bring the second part 54 into contact with the cylindrical member 10 before the first part 53 such that the center axis CX2 of the covering member 50 is parallel to the center axis CXI of the cylindrical member 10. In this way, it is possible to fit the covering member 50 to the cylindrical member 10. In the fitting method described above, the area of the inner circumferential surface of the covering member 50 which slides against the outer circumferential surface of the cylindrical member 10 in the process of moving the covering member 50 is limited to a part of the inner circumferential surface in the vicinity of the first part 53. Hence, it is possible to reduce an area in which friction occurs between the inner circumferential surface of the covering member 50 and the outer circumferential surface of the cylindrical member 10 at the time of fitting, and thus it is possible to reduce a force necessary for fitting the covering member 50. Therefore, it is possible to easily attach and detach the covering member 50, with the result that workability is able to be enhanced.

The covering member 50 includes, in the inner circumferential surface, the step portion 55 formed by the difference between the inside diameter D3 of the first cylindrical portion 51 and the inside diameter D1 of the second cylindrical portion 52. In this way, when the covering member 50 is fitted to the cylindrical member 10, the opening end portion 11a is able to be caused to abut on the step portion 55, and thus it is possible to easily position the covering member 50.

The covering member 50 is flexible, and the thickness of the first cylindrical portion 51 in the radial direction is less than the thickness of the second cylindrical portion 52 in the radial direction. In this way, it is possible to maintain stability when the covering member 50 is fitted to the cylindrical member 10 and is used as the container with the culture membrane 12 serving as the bottom surface and to easily attach and detach the covering member 50 to and from the cylindrical member 10 with the first cylindrical portion 51 having satisfactory flexibility.

The inside diameter D1 of the body cylindrical portion 11 is equal to the inside diameter D1 of the second cylindrical portion 52. Hence, when cells are cultured on both surfaces of the culture membrane 12. the area over which cells are cultured on the first surface 12a and the area over which cells are cultured on the second surface 12b are able to be made equal to each other.

The second cylindrical portion 52 includes the groove portions 56 which are provided in the outer circumferential surface to extend in the circumferential direction. Hence, when the grasping device 80 is used to fit the cylindrical member 10, the convex portions 83 in the grasping device 80 are fitted into the groove portions 56, and thus it is possible to stably grasp the cylindrical member 10. Even when tweezers are used instead of the grasping device 80, the tips of the tweezers are able to be hooked on the groove portions 56, and thus it is possible to stably grasp the cylindrical member 10.

The grasping device 80 includes a pair of grasping portions 81, 81 which are opposite each other through the second cylindrical portion 52 in the radial direction when grasping the second cylindrical portion 52 and each of which includes the grasp inner surface 81a along the outer circumferential surface of the second cylindrical portion 52. In this way, the grasp inner surfaces 81a of the pair of grasping portions 81, 81 are able to be along the outer circumferential surface of the second cylindrical portion 52, and thus it is possible to stably grasp the cylindrical member 10 with the grasping device 80.

### B. Second Embodiment

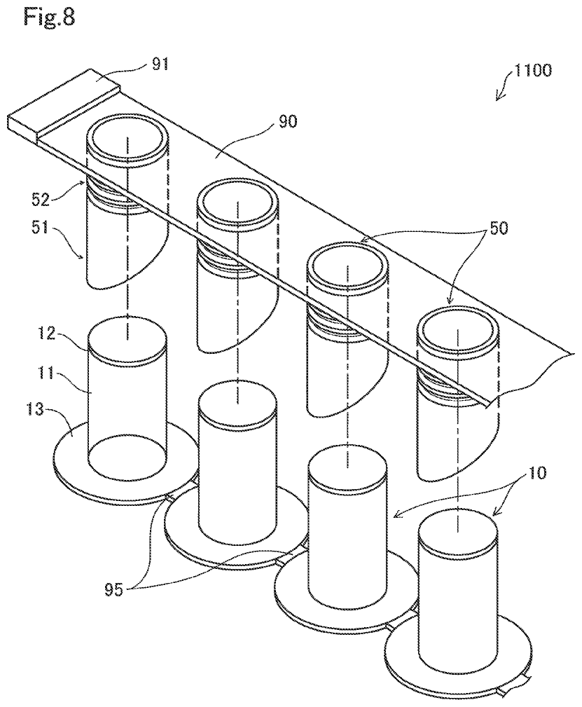

FIG. 8 is a perspective view of a cell culture apparatus 1100 according to a second embodiment. The cell culture apparatus 1100 according to the present embodiment differs from the cell culture apparatus 100 according to the first embodiment in that the cell culture apparatus 1100 includes a holding portion 90 which holds a plurality of covering members 50, coupling portions 95 which couple a plurality of cylindrical members 10 and knobs 91. The same configurations as in the first embodiment are identified with the same symbols, and detailed description thereof are omitted as necessary.

The holding portion 90 is a plate-shaped member that extends in a direction in which a plurality of covering members 50 are aligned. In the holding portion 90, a plurality of holes which penetrate in the direction of thickness are formed. The covering members 50 are respectively inserted into the holes formed in the holding portion 90, and are fixed in the holding portion 90 in a state where end portions of the second cylindrical portions 52 are exposed from the holding portion 90. The plurality of cylindrical members 10 which are aligned on the same straight line are coupled by the coupling portions 95. The intervals of the arrangement of the cylindrical members 10 are the same as the intervals of the arrangement of the covering members 50. Since the covering members 50 are held by the holding portion 90, the covering members 50 are able to be simultaneously fitted to the plurality of cylindrical members 10 aligned on the same straight line. The knobs 91 are attached to both end portions of the holding portion 90 in the direction of a long side. In this way, when the operator attaches and detaches the covering members 50 to and from the cylindrical members 10, the operator holds the knobs 91 with the gloved hands to be able to attach and detach the covering members 50 without directly touching the covering members 50, with the result that it is possible to suppress contamination in the cell culture step.

In the second embodiment described above, the cell culture apparatus 1100 includes the holding portion 90 which holds the covering members 50. In this way, it is possible to simultaneously fit the covering members 50 to the cylindrical members 10. Since the operator holds the holding portion 90 with the hands without touching the covering members 50 to be able to move the covering members 50, it is possible to suppress contamination in the cell culture step.

### C. Other Embodiments

(C1) In the first embodiment described above, when the end surface of the first cylindrical portion 51 of the covering member 50 is seen in the radial direction, a line which is inclined with respect to the center axis CX2 is drawn, and the shape of the end surface is an ellipse. The shape of the end surface of the first cylindrical portion 51 is not limited to an ellipse. For example, when the end surface of the first cylindrical portion 51 of the covering member 50 is seen in the radial direction, the end surface may be a curved line which includes a plurality of inflection points on the way. Regardless of the shape of the end surface, the first cylindrical portion 51 includes the first part 53 and the second part 54, and thus it is possible to easily attach and detach the covering member 50 to and from the cylindrical member 10.

(C2) Although in the description of the first embodiment, the covering member 50 is fitted to the cylindrical member 10 by the operator, the covering member 50 may be mechanically fitted to the cylindrical member 10. Even when the covering member 50 is mechanically fitted, it is possible to reduce a force necessary for fitting the covering member 50, and thus it is possible to easily attach and detach the covering member 50, with the result that workability is able to be enhanced.

The present disclosure is not limited to the embodiments described above, and may be realized in various configurations without departing from the spirit thereof. For example, the technical features of any of the above embodiments and their modifications may be replaced or combined appropriately, in order to solve part or all of the problems described above or in order to achieve part or all of the advantageous effects described above. When the technical features are not described as essential features in the present specification, they may be deleted as necessary. For example, the present disclosure may be realized in embodiments below.

(1) According to one embodiment of the present disclosure, a cell culture apparatus is provided. The cell culture apparatus includes: a cylindrical member that includes a body cylindrical portion and a culture membrane arranged at an opening end portion of the body cylindrical portion; and a cylindrical covering member that is fitted to the body cylindrical portion and includes (i) a first cylindrical portion which covers the outer circumferential surface of the body cylindrical portion when fitted to the cylindrical member and (ii) a second cylindrical portion which is adjacent to the first cylindrical portion in an axial direction of the covering member and forms a container with the culture membrane serving as a bottom surface when fitted to the cylindrical member, and the first cylindrical portion includes a first part in which a length in the axial direction is a first length and a second part in which the length is a second length shorter than the first length. In the embodiment described above, the first cylindrical portion includes the second part and the first part the lengths of which are different from each other. Hence, the covering member is moved with a part near the second part serving as a fulcrum from a posture where the center axis of the covering member is inclined with respect to the center axis of the body cylindrical portion to bring the second part into contact with the cylindrical member before the first part such that the center axis of the covering member is parallel to the center axis of the cylindrical member. In this way, it is possible to fit the covering member to the cylindrical member. In the fitting method described above, an area of the inner circumferential surface of the covering member which slides against the outer circumferential surface of the cylindrical member in the process of moving the covering member is limited to a part of the inner circumferential surface in the vicinity of the first part. Hence, it is possible to reduce, as compared with a case where a covering member having the same length in an axial direction over a circumferential direction is fitted, an area in which friction occurs between the inner circumferential surface of the covering member and the outer circumferential surface of the cylindrical member at the time of fitting. Therefore, it is possible to reduce a force necessary for attaching and detaching the covering member, and thus it is possible to easily attach and detach the covering member, with the result that workability is able to be enhanced.

(2) In the cell culture apparatus of the embodiment described above, the inside diameter of the first cylindrical portion may be greater than the inside diameter of the second cylindrical portion, and the covering member may further include, in a boundary between the inner circumferential surface of the first cylindrical portion and the inner circumferential surface of the second cylindrical portion, a step portion that is formed by a difference between the inside diameter of the first cylindrical portion and the inside diameter of the second cylindrical portion. In this embodiment, when the covering member is fitted to the cylindrical member, the opening end portion of the cylindrical member is able to be caused to abut on the step portion, and thus it is possible to easily position the covering member.

(3) In the cell culture apparatus of the embodiment described above, the covering member may be flexible, and the thickness of the first cylindrical portion in a radial direction may be less than the thickness of the second cylindrical portion in the radial direction. In this embodiment, it is possible to maintain stability when the covering member is used as a container with the culture membrane serving as a bottom surface and to easily attach and detach the covering member to and from the cylindrical member with the first cylindrical portion having satisfactory flexibility.

(4) In the cell culture apparatus of the embodiment described above, the inside diameter of the body cylindrical portion may be equal to the inside diameter of the second cylindrical portion. In this embodiment, when cells are cultured on both surfaces of the culture membrane, the area over which cells are cultured on one surface and the area over which cells are cultured on the other surface are able to he made equal to each other.

(5) In the cell culture apparatus of the embodiment described above, the cell culture apparatus may include: a groove portion that is provided in the outer circumferential surface of the second cylindrical portion to extend in a circumferential direction. In this embodiment, when a device is used to attach and detach the cylindrical member, the tips of the device are able to be hooked on the groove portion, and thus it is possible to stably grasp the cylindrical member.

(6) in the cell culture apparatus of the embodiment described above, the cell culture apparatus may include: a plurality of covering members each being the covering member; and a holding portion that holds the plurality of covering members. In this embodiment, it is possible to simultaneously fit the covering members to the cylindrical members. Since an operator holds the holding portion with the hands without touching the covering members to be able to move the covering members, it is possible to suppress contamination in the cell culture step.

(7) In the cell culture apparatus of the embodiment described above the cell culture apparatus may further include: a grasping device that is provided to sandwich the second cylindrical portion and includes a pair of grasping portions which are opposite each other through the second cylindrical portion in a radial direction when grasping the second cylindrical portion and each of which includes a grasp inner surface along the outer circumferential surface of the second cylindrical portion. In this embodiment, the grasp inner surfaces of the pair of grasping portions are able to be along the outer circumferential surface of the second cylindrical portion, and thus it is possible to stably grasp the cylindrical member with the grasping device.

The present disclosure may be realized by various embodiments, and may be realized, in addition to the embodiments described above, by embodiments of a cell culture method and the like.

What is claimed is:

1. A cell culture apparatus comprising:
a cylindrical member that includes a body cylindrical portion and a culture membrane arranged at an opening end portion of the body cylindrical portion; and
a cylindrical covering member that is fitted to the body cylindrical portion and includes
(i) a first cylindrical portion which covers an outer circumferential surface of the body cylindrical portion when fitted to the cylindrical member and
(ii) a second cylindrical portion which is adjacent to the first cylindrical portion in an axial direction of the covering member and forms a container with the culture membrane serving as a bottom surface when fitted to the cylindrical member,
wherein the first cylindrical portion includes
a first part in which a length in the axial direction is a first length and
a second part in which the length is a second length shorter than the first length.

2. The cell culture apparatus according to claim 1,
wherein an inside diameter of the first cylindrical portion is greater than an inside diameter of the second cylindrical portion, and
the covering member further includes, in a boundary between an inner circumferential surface of the first cylindrical portion and an inner circumferential surface of the second cylindrical portion, a step portion that is formed by a difference between the inside diameter of the first cylindrical portion and the inside diameter of the second cylindrical portion.

3. The cell culture apparatus according to claim 1,
wherein the covering member is flexible, and
a thickness of the first cylindrical portion in a radial direction is less than a thickness of the second cylindrical portion in the radial direction.

4. The cell culture apparatus according to claim 1,
wherein an inside diameter of the body cylindrical portion is equal to an inside diameter of the second cylindrical portion.

5. The cell culture apparatus according to claim 1 further comprising:
a groove portion that is provided in an outer circumferential surface of the second cylindrical portion to extend in circumferential direction.

6. The cell culture apparatus according to claim 1 comprising:
a plurality of covering members each being the covering member; and
a holding portion that holds the plurality of covering members.

7. The cell culture apparatus according to claim 1 further comprising:
a grasping device that is provided to sandwich the second cylindrical portion and includes a pair of grasping portions which are opposite each other through the second cylindrical portion in a radial direction when grasping the second cylindrical portion and each of which includes a grasp inner surface along an outer circumferential surface of the second cylindrical portion.
